(19) United States
(12) Patent Application Publication
(10) Pub. No.: US 2008/0076811 A1
(43) Pub. Date: Mar. 27, 2008

(54) COMBINATIONS COMPRISING DEPEPTIDYPEPTIDASE-IV INHIBITORS AND ANTIDIABETIC AGENTS

(76) Inventors: Bork Balkan, Madison, CT (US); Thomas Edward Hughes, Concord, MA (US); David Grenville Holmes, Binningen (CH); Edwin Bernard Villhauer, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/868,129

(22) Filed: Oct. 5, 2007

Related U.S. Application Data
(63) Continuation of application No. 10/181,169, filed on Oct. 10, 2002, now abandoned.
Application No. 10/181,169 is a continuation-in-part of application No. 09/619,262, filed on Jul. 19, 2000, now abandoned.
(60) Provisional application No. 60/325,744, filed on Jan. 21, 2000.

(51) Int. Cl. A61K 31/427; A61K 31/40; A61P 3/04; A61P 3/10

(52) U.S. Cl. 514/369; 514/408

(57) ABSTRACT

The invention relates to a combination which comprises a DPP-IV inhibitor and at least one further antidiabetic compound, preferably selected from the group consisting of insulin signalling pathway modulators, like inhibitors of protein tyrosine phosphatases (PTPases), non-small molecule mimetic compounds and inhibitors of glutamine-fructose-6-phosphate amidotransferase (GFAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, for simultaneous, separate or sequential use in the prevention, delay of progression or treatment of conditions mediated by dipeptidylpeptidase-IV (DPP-IV), in particular diabetes, more especially type 2 diabetes mellitus, conditions of impaired glucose tolerance (IGT), conditions of impaired fasting plasma glucose, metabolic acidosis, ketosis, arthritis, obesity and osteoporosis; and the use of such combination for the cosmetic treatment of a mammal in order to effect a cosmetically beneficial loss of body weight.

The invention relates to a combination, such as a combined preparation or pharmaceutical composition, respectively, which comprises a dipeptidylpeptidase-IV (DPP-IV) inhibitor and at least one further antidiabetic compound, preferably selected from the group consisting of insulin signalling pathway modulators, like inhibitors of protein tyrosine phosphatases (PTPases), non-small molecule mimetic compounds and inhibitors of glutamine-fructose-6-phosphate amidotransferase (GFAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, for simultaneous, separate or sequential use, especially in the prevention, delay of progression or treatment of conditions mediated by dipeptidylpeptidase-IV (DPP-IV), in particular diabetes, more particular type 2 diabetes mellitus, conditions of impaired glucose tolerance (IGT), conditions of impaired fasting plasma glucose, metabolic acidosis, ketosis, arthritis, obesity and osteoporosis; the use of such combination for the preparation of a pharmaceutical preparation for the prevention, delay of progression or treatment of such conditions; the use of such combination for the cosmetic treatment of a mammal in order to effect a cosmetically beneficial loss of body weight; a method of prevention, delay of progression or treatment of conditions mediated by DPP-IV; a method of improving the bodily appearance of a warm-blooded animal.

DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

Non-insulin dependent diabetes mellitus (type 2 diabetes mellitus) is characterized by both increased peripheral insulin resistance and abnormal insulin secretion. At least three abnormalities of insulin secretion are recognized: in the first phase, insulin secretion is lost and in the second phase insulin is both delayed and inadequate in the face of elevated circulating glucose levels. Several metabolic, hormonal, and pharmacological entities are known to stimulate insulin secretion including glucose, amino-acids and gastrointestinal peptides. The Diabetes Control and Complications Trial (DCCT) has established that lowering of blood glucose is associated with decreases in the onset and progression of diabetic microvascular complications (Diabetes Control and Complications Trial Research Group; N. Engl. J. Med. 1993, 329, 977-986). IGT is an impairment of glucose homeostasis closely related to type 2 diabetes mellitus. Both conditions convey a great risk of macrovascular disease. Therefore, one therapeutic focus is on optimizing and potentially normalizing glycemic control in subjects with type 2 diabetes mellitus, conditions of impaired fasting plasma glucose, or IGT. Presently available agents need to be improved in order to better meet this therapeutic challenge.

The present invention relates to a combination which comprises a DPP-IV inhibitor in free or pharmaceutically acceptable salt form, and at least one further antidiabetic compound or the pharmaceutically acceptable salt of such a compound and optionally at least one pharmaceutically acceptable carrier; for simultaneous, separate or sequential use.

Preferably, the antidiabetic compound is selected from the group consisting of insulin signalling pathway modulators, like inhibitors of protein tyrosine phosphatases (PTPases), non-small molecule mimetic compounds and inhibitors of glutamine-fructose-6-phosphate amidotransferase (GFAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or the pharmaceutically acceptable salts of such a compound and optionally at least one pharmaceutically acceptable carrier; for simultaneous, separate or sequential use, particularly in the prevention, delay of progression or treatment of conditions mediated by DPP-IV, in particular conditions of impaired glucose tolerance (IGT), conditions of impaired fasting plasma glucose, metabolic acidosis, ketosis, arthritis, obesity and osteoporosis, and preferably diabetes, especially type 2 diabetes mellitus. Such a combination is preferably a combined preparation or a pharmaceutical composition.

The DPP-IV inhibitor can be peptidic or non-peptidic. Preferably, the DPP-IV inhibitor is non-peptidic.

Unless stated otherwise in the present disclosure organic radicals designated “lower” contain not more than 7, preferably not more than 4, carbon atoms and the following expressions have the meanings as given below:

Halogen represents preferably fluoro, chloro or bromo.

Lower alkyl is, if not stated otherwise, preferably ethyl or, most preferably, methyl. (C₁₋₈)Alkyl is branched or preferably unbranched alkyl, preferably lower alkyl, e.g. methyl or ethyl.

Lower alkylene is preferably methylene, ethylene or propylene. It can be unsubstituted or substituted e.g. by hydroxy.

Lower alkoxy is preferably methoxy or ethoxy. (C₂₋₄)Alkoxy is e.g. ethoxy or propoxy.

Cycloalkyl is e.g. C₃-C₁₂cycloalkyl, preferably cyclopropyl, cyclobutyl, cyclopentyl, cyclohexyl, cycloheptyl or cyclodecyl; or bicycloalkyl such as bicycloheptyl. Cycloalkenyl is preferably 1-cyclohexenyl, 2-cyclohexenyl, 3-cyclohexenyl, 1-cyclopentenyl or 1-cyclopentenyl.

(C₁₋₃)Hydroxyalkyl is e.g. 3-hydroxypropyl, 1-hydroxyethyl or hydroxymethyl.

C₄-C₆-Alkylenimino which is unsubstituted or substituted by one or two lower alkyl groups is, for example, pyrrolidinyl, methylpyrrolidinyl, 1-piperidinyl, 2-piperidinyl, 3-piperidinyl, 2-methyl-1-piperidinyl or hexamethylenimino. Preferably, C₄-C₆-alkylenimino is 1-piperidinyl.

A [3.1.1]bicyclic carbocyclic moiety optionally substituted as defined above preferably is bicyclo[3.1.1]hept-2-yl optionally disubstituted in 6-position with methyl, or bicyclo[3.1.1]-hept-3-yl optionally trisubstituted with one methyl in 2-position and two methyl groups in 6-position. A [2.2.1]bicyclic carbocyclic moiety optionally substituted as defined above preferably is bicyclo[2.2.1]hept-2-yl.

Aryl comprises preferably 6 to 12 carbon atoms and is e.g. phenyl, tolyl or naphthyl, each of which can be substituted e.g. by lower alkyl or halogen.

The term “heteroaryl” refers to an aromatic heterocyclic radical selected, for example, from the group consisting of pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, azepinyl, 4-piperidinyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane, indolyl, benzothiazolyl, benzoxazolyl, benzothienyl, quinuclidinyl, quinolinyl, tetrahydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl, dihydrobenzoisothiazolyl, dihydroisoindolyl, dihydroquinazolinyl and tetrahydroquinazolinyl.

Preferred DPP-IV inhibitors are N—(N′-substituted glycyl)-2-cyanopyrrolidines represented by formula (I), 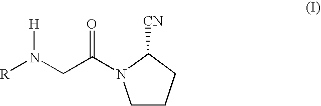
wherein R is:
a) R₁R₁ₐN(CH₂)ₘ— wherein
   R₁ is a pyridinyl or pyrimidinyl moiety optionally mono- or independently disubstituted with lower alkyl, lower alkoxy, halogen, trifluoromethyl, cyano or nitro; or phenyl optionally mono- or independently disubstituted with lower alkyl, lower alkoxy or halogen;
   R₁ₐ is hydrogen or (C₁₋₈)alkyl; and
   m is 2 or 3;
b) (C₃₋₁₂)cycloalkyl optionally monosubstituted in the 1-position with (C₁₋₃)hydroxyalkyl;
c) R₂(CH₂)ₙ— wherein either
   R₂ is phenyl optionally mono- or independently di- or independently trisubstituted with lower alkyl, lower alkoxy, halogen or phenylthio optionally monosubstituted in the phenyl ring with hydroxymethyl; or is (C₁₋₈)alkyl; a [3.1.1]bicyclic carbocyclic moiety optionally mono- or plurisubstituted with (C₁₋₈)alkyl; a pyridinyl or naphthyl moiety optionally mono- or independently disubstituted with lower alkyl, lower alkoxy or halogen; cyclohexene; or adamantyl; and
   n is 1 to 3; or
   R₂ is phenoxy optionally mono- or independently disubstituted with lower alkyl, lower alkoxy or halogen; and
   n is 2 or 3;
d) (R₃)₂CH(CH₂)₂— wherein each R₃ independently is phenyl optionally mono- or independently disubstituted with lower alkyl, lower alkoxy or halogen;
e) R₄(CH₂)ₚ— wherein R₄ is 2-oxopyrrolidinyl or (C₂₋₄)alkoxy and
   p is 2 to 4;
f) isopropyl optionally monosubstituted in 1-position with (C₁₋₃)hydroxyalkyl;
g) R₅ wherein R₅ is: indanyl; a pyrrolidinyl or piperidinyl moiety optionally substituted with benzyl; a [2.2.1]- or [3.1.1]bicyclic carbocyclic moiety optionally mono- or plurisubstituted with (C₁₋₈)alkyl; adamantyl; or (C₁₋₈)alkyl optionally mono- or independently plurisubstituted with hydroxy, hydroxymethyl or phenyl optionally mono- or independently disubstituted with lower alkyl, lower alkoxy or halogen;
h) a substituted adamantyl
   in free form or in acid addition salt form.

In a preferred embodiment of the invention, the N—(N′-substituted glycyl)-2-cyanopyrrolidine is represented by formula (I), wherein
R is R₁R₁ₐN(CH₂)ₘ— wherein
   R₁ is a pyridinyl or pyrimidinyl moiety optionally mono- or independently disubstituted with lower alkyl, lower alkoxy, halogen, trifluoromethyl, cyano or nitro; or phenyl optionally mono- or independently disubstituted with lower alkyl, lower alkoxy or halogen;
   R₁ₐ is hydrogen or (C₁₋₈)alkyl; and
   m is 2 or 3;
      in free form or in acid addition salt form.

More preferably, the N—(N′-substituted glycyl)-2-cyanopyrrolidine is represented by formula (I), wherein
R is R₁R₁ₐN(CH₂)ₘ— wherein
   R₁ is a pyridinyl moiety optionally mono- or independently disubstituted with lower alkyl, lower alkoxy, halogen, trifluoromethyl, cyano or nitro;
   R₁ₐ is hydrogen or (C₁₋₈)alkyl; and
   m is 2 or 3;
      in free form or in acid addition salt form.

Most preferably, the N—(N′-substituted glycyl)-2-cyanopyrrolidine of formula I is (S)-1-{2-[5-cyanopyridin-2-yl)amino]ethyl-aminoacetyl}-2-cyano-pyrrolidine (DPP728) or (S)-1-[(3-hydroxy-1-adamantyl)amino]acetyl-2-cyano-pyrrolidine (LAF237).

In another preferred embodiment, the DPP-IV inhibitor is selected from the compounds of formulae II, III, IV and V: 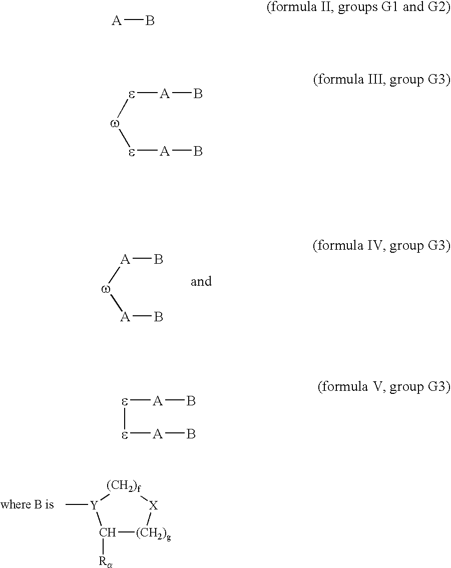
g is 0, 1 or 2;
X is CH₂, O, S, SO, SO₂, NH or NRα₁ where Rα₁ is lower alkyl (C₁ to C₆);
—Y is —N, —CH or —C═ (when the —CO group of A is replaced with —CH═ or —CF═);
Rα is H, CN, CHO, B(OH)₂, PO₃H or an ester thereof, CC—Rα₇, or CH═N—Rα₈ where Rα₇ is H, F, lower alkyl (C₁ to C₆), CN, NO₂, ORα₉, CO₂Rα₉ or CORα₉; Rα₉ is lower alkyl (C₁ to C₆); Rα₈ is Ph, OH, ORα₉, OCORα₉ or OBn; A is attached to Y;
   and wherein for the group G1 compounds
(a) when Rα is H, A is an α-amino-acyl group derived from an β-amino-acid bearing a cycloaliphatic side-chain or is a β-amino-acyl group of general formula 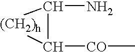
   where h is 1 to 6, the ring in either case optionally having unsaturation and/or heteroatom substitution;
(b) when Rα is CN, CC—Rα₇, or CH═N—Rα₈, A is as defined at (a) and in addition may be derived from any L-α-amino acid bearing a lipophilic side-chain;
(c) and when Rα is CHO or B(OH)₂, A is a β-amino-acyl group as defined under (a);
   for the group G2 compounds, Rα is H, CN, C═C—Rα₇ or —CH═N—Rα₈ and A is 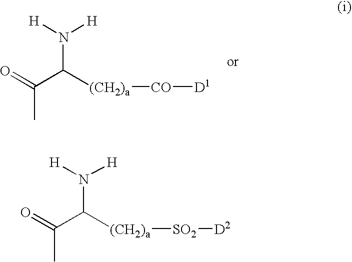
   where a is 1-5; D¹ is -G-(CH₂)_{b}—(Rα₄)_{q}—Rα₃; G is O, NH or NMe; b is 0-12; q is 0-5; D² is D¹ with G≠O; Rα₄ is Z-NH—(CH₂)_{c}— or NH-Z-(CH₂)_{c}— where c is 1-12 and Z is CO, CH₂ or SO₂; Rα₃ is CO₂H or an ester thereof, CONH₂, CONHNH₂, CONRα₅Rα₆, CONHNRα₅Rα₆, PO₃H or an ester thereof, SO₃H, SO₂NH₂, SO₂NRα₅Rα₆, OH, ORα₅, substituted or unsubstituted aryl or heteroaryl, NH₂, NRα₅Rα₆, NHCO₂Rα₅, NHSO₂NRα₅Rα₆, NHCORα₅, NH—SO₂Rα₅, NH—CH(:NRα₅)NRα₅Rα₆, NHCONHRα₅Rα₆, sugar, CO-aminosugar, NHCO-aminosugar or —NHCS-aminosugar; and Rα₅ and Rα₆ are independently selected from H and lower alkyl, fluoroalkyl and cycloalkyl group of up to 8 atoms and aryl, heteroaryl and alkyl heteroaryl groups of up to 11 atoms or Rα₅ and Rα₆ may together comprise a chain (C₃ to C₈); or is 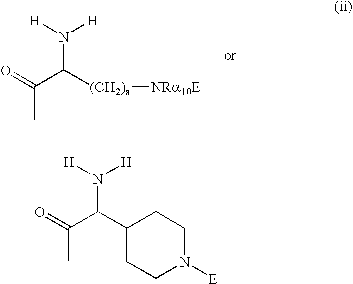
      where Rα₁₀ is H or Me, the ring may contain more heteroatoms, E is J-(CH₂)_{b}—(Rα₄)_{q}—Rα₃, J=CO, CH₂ or SO₂, and a, b, q, Rα₃ and Rα₄ are as defined under (i); or is 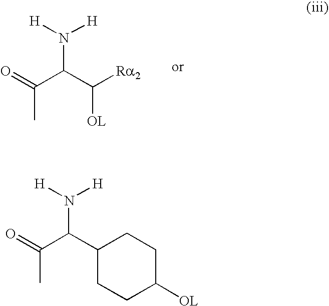
      where Rα₂ is H or Me, the ring may contain one or more heteroatoms, and L is (CH₂)_{d}—(CO)ᵣ—(CH₂)_{b}—(Rα₄)_{q}—Rα₃ or (CH₂)ₑ—NRα₁₀—(CH₂)_{b}—(Rα₄)_{q}—Rα₃ where r is 0 or 1, d is 0-4, e is 2-4, and b, q, Rα₃ and Rα₄ are as defined under (i);
      and for the group G3 compounds, each B may have any identity defined therefor above, each A may be chosen from any group G2 structure (i), (ii) or (iii) above with the terminal groups Rα₃ in the A residues replaced with a shared group -ε-ω-ε- or -ε-ε- or -ω-, and ε and ω are selected independently from CH₂, O, NH, CO, S, SO₂, Ph and NHMe;
      and wherein in groups G2 and G3 at least one CH₂ group in a chain may be replaced by a bioisostere thereof or any amide group which connects A and B in a group G1, G2 or G3 compound or which is in a side-chain of A in a Group G2 or G3 compound may be replaced by an amide bioisostere,
      in free form or in acid addition salt form.

In another preferred embodiment, the DPP-IV inhibitor is a compound of formula VI 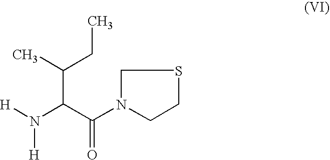
in free form or in acid addition salt form.

DPP-IV inhibitors are in each case generically and specifically disclosed in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications. DPP728 and LAF237 are specifically disclosed in Example 3 of WO 98/19998 and Example 1 of WO 00/34241, respectively. A DPP-IV inhibitor of formula VI (see above) is specifically described in Diabetes 1998, 47, 1253-1258. DPP728 can be formulated as described on page 20 of WO 98/19998.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula VII 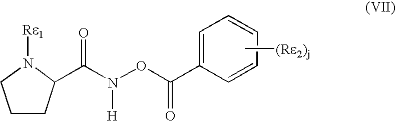
wherein
j is 0, 1 or 2;
Rε₁ represents the side chain of a natural amino acid; and
Rε₂ represents lower alkoxy, lower alkyl, halogen or nitro;
   or a pharmaceutically acceptable salt thereof.

In a very preferred embodiment of the invention, the N-peptidyl-O-aroyl hydroxylamine is a compound of formula VIIa 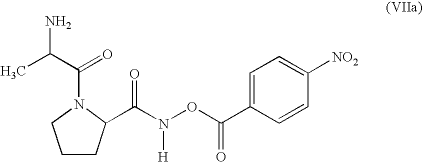
or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Examples of “inhibitors of PTPase” include, but are not limited to those disclosed in U.S. Pat. No. 6,057,316, U.S. Pat. No. 6,001,867, WO 99/58518, WO 99/58522, WO 99/46268, WO 99/46267, WO 99/46244, WO 99/46237, WO 99/46236, WO 99/15529 and by Poucheret et al in Mol. Cell. Biochem. 1998, 188, 73-80.

Examples of “non-small molecule mimetic compounds” include, but are not limited to those disclosed in Science 1999, 284; 974-97, especially L-783,281, and WO 99/58127, especially CLX-901.

Examples of “inhibitors of GFAT” include, but are not limited to those disclosed in Mol. Cell. Endocrinol. 1997, 135(1), 67-77.

The term “inhibitors of G6Pase” used herein means a compound or composition which reduces or inhibits hepatic gluconeogenesis by decreasing or inhibiting the activity of G6Pase. Examples of such compounds are disclosed in WO 00/14090, WO 99/40062, WO. 98/40385, EP682024 and Diabetes 1998, 47, 1630-1636.

The term “inhibitors of F-1,6-BPase” used herein means a compound or composition which reduces or inhibits hepatic gluconeogenesis by decreasing or inhibiting the activity of F-1,6-BPase. Examples of such compounds are disclosed in WO 00/14095, WO 99/47549, WO 98/39344, WO 98/39343 and WO 98/39342.

The term “inhibitors of GP” used herein means a compound or composition which reduces or inhibits hepatic glycogenolysis by decreasing or inhibiting the activity of GP. Examples of such compounds are disclosed in EP 978279, U.S. Pat. No. 5,998,463, WO 99/26659, EP 846464, WO 97/31901, WO 96/39384, WO9639385 and in particular CP-91149 as described in Proc. Natl. Acad Sci USA 1998, 95, 1776-1781.

The term “glucagon receptor antagonists” as used herein relates in particular to the compounds described in WO 98/04528, especially BAY27-9955, and those described in Bioorg Med. Chem. Lett 1992, 2, 915-918, especially CP-99,711, J. Med. Chem. 1998, 41, 5150-5157, especially NNC 92-1687, and J. Biol. Chem. 1999, 274; 8694-8697, especially L-168,049 and compounds disclosed in U.S. Pat. No. 5,880,139, WO 99/01423, U.S. Pat. No. 5,776,954, WO 98/22109, WO 98/22108, WO 98/21957 and WO 97/16442.

The term “inhibitors of PEPCK” used herein means a compound or composition which reduces or inhibits hepatic gluconeogenesis by decreasing or inhibiting the activity of PEPCK. Examples of such compounds are disclosed in U.S. Pat. No. 6,030,837 and Mol. Biol. Diabetes 1994, 2, 283-99.

The term “PDHK inhibitors” as used herein means inhibitors of pyruvate dehydrogenase kinase and include, but are not limited to, those compounds disclosed by Aicher et al in J. Med. Chem. 42 (1999) 2741-2746.

The term “insulin sensitivity enhancer” used herein means any and all pharmacological active compounds that enhance the tissue sensitivity towards insulin. Insulin sensitivity enhancers include, e.g., inhibitors of GSK-3, retinoid X receptor (RXR) agonists, agonists of Beta-3 AR, agonists of UCPs, antidiabetic thiazolidinediones (glitazones), non-glitazone type PPARγ agonists, dual PPARγ/PPARα agonists, antidiabetic vanadium containing compounds and biguanides, e.g., metformin.

The insulin sensitivity enhancer is preferably selected from the group consisting of antidiabetic thiazolidinediones, antidiabetic vanadium containing compounds and metformin.

In one preferred embodiment, the insulin sensitivity enhancer is metformin.

Examples of “inhibitors of GSK-3” include, but are not limited to those disclosed in WO 00/21927 and WO 97/41854.

By “RXR agonist” is meant a compound or composition which when combined with RXR homodimers or heterodimers increases the transcriptional regulation activity of RXR, as measured by an assay known to one skilled in the art, including, but not limited to, the “co-transfection” or “cis-trans” assays described or disclosed in U.S. Pat. Nos. 4,981,784, 5,071,773, 5,298,429, 5,506,102, WO89/05355, WO91/06677, WO92/05447, WO93/11235, WO95/18380, PCT/US93/04399, PCT/US94/03795 and CA 2,034,220, which are incorporated by reference herein. It includes, but is not limited to, compounds that preferentially activate RXR over RAR (i.e. RXR specific agonists), and compounds that activate both RXR and RAR (i.e. pan agonists). It also includes compounds that activate RXR in a certain cellular context but not others (i.e. partial agonists). Compounds disclosed or described in the following articles, patents and patent applications which have RXR agonist activity are incorporated by reference herein: U.S. Pat. Nos. 5,399,586 and 5,466,861, WO96/05165, PCT/US95/16842, PCT/US95/16695, PCT/US93/10094, WO94/15901, PCT/US92/11214, WO93/11755, PCT/US93/10166, PCT/US93/10204, WO94/15902, PCT/US93/03944, WO93/21146, provisional applications 60,004,897 and 60,009,884, Boehm, et al. J. Med. Chem. 38(16):3146-3155, 1994, Boehm, et al. J. Med. Chem. 37(18):2930-2941, 1994, Antras et al., J. Biol. Chem. 266:1157-1161 (1991), Salazar-Olivo et al., Biochem. Biophys. Res. Commun. 204:157-263 (1994) and Safanova, Mol. Cell. Endocrin. 104:201-211 (1994). RXR specific agonists include, but are not limited to, LG 100268 (i.e. 2-[1-(3,5,5,8,8-pentamethyl-5,6,7,8-tetrahydro-2-naphthyl)-cyclopropyl]-pyridine-5-carboxylic acid) and LGD 1069 (i.e. 4-[(3,5,5,8,8-pentamethyl-5,6,7,8-tetrahydro-2-naphthyl)-2-carbonyl]-benzo ic acid), and analogs, derivatives and pharmaceutically acceptable salts thereof. The structures and syntheses of LG 100268 and LGD 1069 are disclosed in Boehm, et al. J. Med. Chem. 38(16):3146-3155, 1994, incorporated by reference herein. Pan agonists include, but are not limited to, ALRT 1057 (i.e. 9-cis retinoic acid), and analogs, derivatives and pharmaceutically acceptable salts thereof.

Examples of “agonists of Beta-3 AR” include, but are not limited to CL-316,243 (Lederle Laboratories) and those disclosed in WO 99/29672, WO 98/32753, WO 98/20005, WO 98/09625, WO 97/46556, WO 97/37646 and U.S. Pat. No. 5,705,515.

The term “agonists of UCPs” used herein means agonists of UCP-1, preferably UCP-2 and even more preferably UCP-3. UCPs are disclosed in Vidal-Puig et al., Biochem. Biophys. Res. Commun., Vol. 235(1) pp. 79-82 (1997). Such agonists are a compound or composition which increases the activity of UCPs.

The antidiabetic thiazolidinedione (glitazone) is, for example, (S)-((3,4-dihydro-2-(phenyl-methyl)-2H-1-benzopyran-6-yl)methyl-thiazolidine-2,4-dione (englitazone), 5-([4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)-methyl]phenyl}methane (YM268), 5-(4-[2-(5-methyl-2-phenyl-4-oxazolyl)-2-hydroxyethoxy]-benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)-naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297).

Preferably, the antidiabetic thiazolidinedione is a compound of formula VIII, 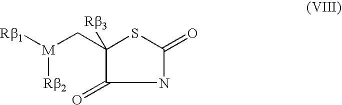
wherein
M represents
   naphthyl, benzoxazolyl, dihydrobenzopyranyl, indole,
   phenyl (optionally substituted by halogen) or phenylethynyl (optionally substituted by halogen);
Rβ₁ represents halogen or a radical -QRβ₄, in which
   Q can be oxygen, lower alkylen, carbonyl or —NH—,
   Rβ₄ is
      naphthyl;
      phenyl, unsubstituted or substituted by 2,4-dioxo-5-thiazolidinyl; or
      lower alkyl or hydroxy lower alkyl, unsubstituted or substituted by
         a) indole or 2,3-dihydroindole,
         b) pyridyl, lower alkyl-pyridyl, N-lower alkyl-N-pyridylamino or halogenphenyl,
         c) dihydrobenzopyranyl, which is unsubstituted or substituted by hydroxy and lower alkyl,
         d) oxazolyl, which is substituted by lower alkyl and phenyl,
         e) cycloalkyl, which is unsubstituted or substituted by lower alkyl, or
         f) arylcycloalkylcarbonyl;
Rβ₂ represents hydrogen or trifluoromethylphenyl-lower alkyl carbamoyl; and
Rβ₃ represents hydrogen or arylsulfonyl;
   or a pharmaceutically acceptable salt thereof.

Preferably, the compound of formula VIII is selected from the group consisting of (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297) or a pharmaceutically acceptable salt thereof.

More preferably, the compound of formula VIII is selected from the group consisting of 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione(pioglitazone) and 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), MCC555, T-174 and KRP297, especially rosiglitazone, pioglitazone and troglitazone, or a pharmaceutically acceptable salt thereof.

The glitazones 5-{[4-(2-(5-ethyl-2-pyridyl)ethoxy)phenyl]-methyl}thiazolidine-2,4-dione (pioglitazone, EP 0 193 256 A1), 5-{[4-(2-(methyl-2-pyridinyl-amino)-ethoxy)phenyl]methyl}-thiazolidine-2,4-dione (rosiglitazone, EP 0 306 228 A1), 5-{[4-((3,4-dihydro-6-hydroxy-2,5,7,8-tetramethyl-2H-1-benzopyran-2-yl)methoxy)-phenyl]-methyl}thiazolidine-2,4-dione (troglitazone, EP 0 139 421), (S)-((3,4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl)benzamide (KRP297, JP 10087641-A), 5-[6-(2-fluoro-benzyloxy)naphthalen-2-ylmethyl]thiazolidine-2,4-dione (MCC555, EP 0 604 983 B1), 5-{[4-(3-(5-methyl-2-phenyl-4-oxazolyl)-1-oxopropyl)-phenyl]-methyl}-thiazolidine-2,4-dione (darglitazone, EP 0 332 332), 5-(2-naphthylsulfonyl)-thiazolidine-2,4-dione (AY-31637, U.S. Pat. No. 4,997,948), 5-([4-(1-methyl-cyclohexyl)methoxy)-phenyl]methyl)-thiazolidine-2,4-dione (ciglitazone, U.S. Pat. No. 4,287,200) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-{[4-(2-(2,3-dihydroindol-1-yl)ethoxy)phenyl]methyl}-thiazolidine-2,4-dione is described in B. B. Lohray et al., J. Med. Chem. 1998, 41, 1619-1630; Examples 2d and 3g on pages 1627 and 1628. The preparation of 5-[3-(4-chlorophenyl])-2-propynyl]-5-phenylsulfonyl)-thiazolidine-2,4-dione and the other compounds in which A is phenylethynyl mentioned herein can be carried out according to the methods described in J. Wrobel et al., J. Med. Chem. 1998, 41, 1084-1091.

In particular, MCC555 can be formulated as disclosed on page 49, lines 30 to 45, of EP 0 604 983 B1; englitazone as disclosed from page 6, line 52, to page 7, line 6, or analogous to Examples 27 or 28 on page 24 of EP 0 207 605 B1; and darglitazone and S-(4-[2-(5-methyl-2-phenyl-4-oxazolyl)-ethoxy)]benzyl)-thiazolidine-2,4-dione (BM-13.1246) can be formulated as disclosed on page 8, line 42 to line 54 of EP 0 332 332 B1. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200.

Non-glitazone type PPARγ agonists are especially N-(2-benzoylphenyl)-L-tyrosine analogues, e.g. GI-262570, and JTT501.

The term “dual PPARγ/PPARα agonists” as used herein means compounds which are at the same time PPARγ and PPARα agonists. Preferred dual PPARγ/PPARα agonists are especially those ω-[(oxoquinazolinylalkoxy)phenyl]alkanoates and analogs thereof, very especially the compound DRF-554158, described in WO 99/08501 and the compound NC-2100 described by Fukui in Diabetes 2000, 49(5), 759-767.

Preferably, the antidiabetic vanadium containing compound is a physiologically tolerable vanadium complex of a bidentate monoprotic chelant, wherein said chelant is an α-hydroxypyrone or α-hydroxypyridinone, especially those disclosed in the Examples of U.S. Pat. No. 5,866,563, of which the working examples are hereby incorporated by reference, or a pharmaceutically acceptable salt thereof.

The preparation of metformin (dimethyldiguanide) and its hydrochloride salt is state of the art and was disclosed first by Emil A. Werner and James Bell, J. Chem. Soc. 121, 1922, 1790-1794. Metformin, can be administered e.g. in the form as marketed under the trademarks GLUCOPHAGE™.

Insulin secretion enhancers are pharmacological active compounds having the property to promote secretion of insulin from pancreatic β cells. Examples for insulin secretion enhancers include glucagon receptor antagonists (see above), sulphonyl urea derivatives, incretin hormones, especially glucagon-like peptide-1 (GLP-1) or GLP-1 agonists, β-cell imidazoline receptor antagonists, and short-acting insulin secretagogues, like antidiabetic phenylacetic acid derivatives, antidiabetic D-phenylalanine derivatives and BTS 67582 described by T. Page et al in Br. J. Pharmacol. 1997, 122, 1464-1468.

The sulphonyl urea derivative is, for example, glisoxepid, glyburide, glibenclamide, acetohexamide, chloropropamide, glibornuride, tolbutamide, tolazamide, glipizide, carbutamide, gliquidone, glyhexamide, phenbutamide or tolcyclamide; and preferably glimepiride or gliclazide. Tolbutamide, glibenclamide, gliclazide, glibornuride, gliquidone, glisoxepid and glimepiride can be administered e.g. in the form as they are marketed under the trademarks RASTINON HOECHST™, AZUGLUCON™, DIAMICRON™, GLUBORID™, GLURENORM™, PRO-DIABAN™ and AMARYL™, respectively.

GLP-1 is a insulinotropic protein which was described, e.g., by W. E. Schmidt et al. in Diabetologia 28, 1985, 704-707 and in U.S. Pat. No. 5,705,483. The term “GLP-1 agonists” used herein means variants and analogs of GLP-1 (7-36)NH₂ which are disclosed in particular in U.S. Pat. No. 5,120,712, U.S. Pat. No. 5,118,666, U.S. Pat. No. 5,512,549, WO 91/11457 and by C. Orskov et al in J. Biol. Chem. 264 (1989) 12826. The term “GLP-1 agonists” comprises especially compounds like GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al in Diabetologia 1999, 42, 45-50.

The term “β-cell imidazoline receptor antagonists” as used herein means compounds as those described in WO 00/78726 and by Wang et al in J. Pharmacol. Exp. Ther. 1996; 278; 82-89, e.g. PMS 812.

The antidiabetic phenylacetic acid derivative is preferably a compound of formula IX 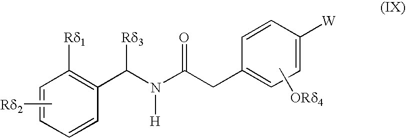
wherein
Rδ₁ is an unbranched C₄-C₆alkyleneimino group which is unsubstituted or mono- or disubstituted by C₁-C₃alkyl;
Rδ₂ is hydrogen, halogen, methyl or methoxy;
Rδ₃ is hydrogen, C₁-C₇alkyl, or phenyl which is unsubstituted or substituted by halogen, methyl or methoxy;
Rδ₄ is hydrogen, allyl, acetyl or propionyl or C₁-C₃alkyl which is unsubstituted or substituted by phenyl; and
W is methyl, hydroxymethyl, formyl, carboxy; or alkoxycarbonyl which comprises between 2 and up to and including 5 carbon atoms and in which the alkyl moiety of the alkoxy group is unsubstituted or substituted by phenyl or a pharmaceutically acceptable salt thereof.

Most preferably, the compound of formula IX is repaglinide or a pharmaceutically acceptable salt thereof.

The antidiabetic D-phenylalanine derivative is preferably a compound of formula X 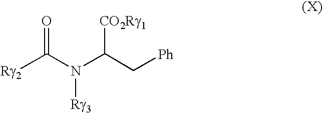
wherein Ph has the meaning of phenyl,
Rγ₁ is selected from hydrogen, C₁ to C₅alkyl, C₆ to C₁₂ aryl, C₆ to C₁₂arylalkyl, 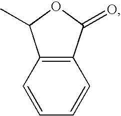
—CH₂CO₂Rγ₃, —CH(CH₃)—OCO—Rγ₃, and —CH₂—OCO—C(CH₃)₃;
Rγ₂ is selected from groups comprising C₆ to C₁₂ aryl, heteroaryl, cycloalkyl, or cycloalkenyl, any of which groups may have one or more substitutents; and
Rγ₃ is selected from hydrogen and C₁ to C₅ alkyl, with the proviso that when Rγ₁ and Rγ₃ are both hydrogen then Rγ₂ is other than substituted or unsubstituted phenyl or naphthyl;
   or a pharmaceutically acceptable salts thereof or a precursor which can be converted thereto in the human or animal body.

If Rγ₂ represents heteroaryl, Rγ₂ is preferably quinolynyl, pyridyl or 2-benzofuranyl.

Most preferably, the antidiabetic D-phenylalanine derivative is nateglinide or a pharmaceutically acceptable salt thereof.

Nateglinide (N-[(trans-4-isopropylcyclohexyl)-carbonyl]-D-phenylalanine, EP 196222 and EP 526171) and repaglinide ((S)-2-ethoxy-4-(2-[[3-methyl-1-[2-(1-piperidinyl)phenyl]butyl]amino]-2-oxoethyl)benzoic acid, EP 0 147 850 A2, in particular Example 11 on page 61, and EP 0 207 331 A1) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The term nateglinide as used herein comprises crystal modifications (polymorphs) such as those disclosed in EP 0526171 B1 or U.S. Pat. No. 5,488,510, respectively, the subject matter of which is incorporated by reference to this application, especially the subject matter of claims 8 to 10 as well as the corresponding references to the B-type crystal modification. Preferably, in the present invention the B- or H-type, more preferably the H-type, is used. Repaglinde can be administered in the form as it is marketed e.g. under the trademark NovoNorm™. Nateglinide can be administered in the form as it is marketed e.g. under the trademark STARLIX™.

α-Glucosidase inhibitors are pharmacological active compounds which inhibit small intestinal α-glucosidase enzymes which break down non-adsorbable complex carbohydrates into absorbable monosaccharides. Examples for such compounds are acarbose, N-(1,3-dihydroxy-2-propyl)valiolamine (voglibose) and the 1-deoxynojirimycin derivative miglitol. Acarbose is 4″,6″-dideoxy-4″-[(1S)-(1,4,6/5)-4,5,6-trihydroxy-3-hydroxymethyl-2-cyclo-hexenylamino}maltotriose. The structure of acarbose can as well be described as O-4,6-dideoxy-4-{[1S,4R,5S,6S]-4,5,6-trihydroxy-3-(hydroxymethyl)-2-cyclohexen-1-yl]-amino}-α-D-glucopyranosyl-(1→4)-O-α-D-glucopyranosyl-(1→4)-D-glucopyranose. Acarbose (U.S. Pat. No. 4,062,950 and EP 0 226 121), is generically and specifically disclosed in the documents cited in brackets, in particular in the compound claims and the final products of the working examples, the subject-matter of the final products, the pharmaceutical preparations and the claims are hereby incorporated into the present application by reference to these publications. Corresponding to the needs of the single patient it can be possible to administer acarbose in the form as it is marketed e.g. under the trademark GLUCOBAY™. Miglitol can be administered in the form as it is marketed e.g. under the trademark DIASTABOL 50™

The α-glucosidase inhibitor is preferably selected from the group consisting of acarbose, voglibose and miglitol.

Examples of “inhibitors of gastric emptying” other than GLP-1 include, but are not limited to those disclosed in J. Clin. Endocrinol. Metab. 2000, 85(3), 1043-1048, especially CCK-8, and in Diabetes Care 1998; 21; 897-893, especially Amylin and analogs thereof, e.g. Pramlintide. Amylin is also described e.g. by O. G. Kolterman et al. in Diabetologia 39, 1996, 492-499.

Examples of “α₂-adrenergic antagonists” include, but are not limited to midaglizole described in Diabetes 36, 1987, 216-220.

Comprised are likewise the corresponding stereoisomers as well as the corresponding polymorphs, e.g. crystal modifications, which are disclosed in the cited patent documents.

In a very preferred embodiment of the invention, the DPP-IV inhibitor is selected from (S)-1-[(3-hydroxy-1-adamantyl)amino]acetyl-2-cyano-pyrrolidine and (S)-1-{2-[5-cyanopyridin-2-yl)amino]ethyl-aminoacetyl}-2-cyano-pyrrolidine, and the further antidiabetic compound is selected from the group consisting of nateglinide, repaglinide, metformin, rosiglitazone, pioglitazone, troglitazone, glisoxepid, glyburide, glibenclamide, acetohexamide, chloropropamide, glibornuride, tolbutamide, tolazamide, glipizide, carbutamide, gliquidone, glyhexamide, phenbutamide, tolcyclamide, glimepiride and gliclazide, or the pharmaceutically acceptable salt of such a compound.

The term “prevention” means prophylactic administration of the combination to healthy patients to prevent the outbreak of the conditions mentioned herein. Moreover, the term “prevention” means prophylactic administration of such combination to patients being in a pre-stage of the conditions, especially diabetes, to be treated.

The term “delay of progression” used herein means administration of the combination, such as a combined preparation or pharmaceutical composition, to patients being in a pre-stage of the condition, especially diabetes, to be treated in which patients a pre-form of the corresponding condition is diagnosed.

Examples of the preparation and formulation of inhibitors of PTPases, inhibitors of GSK-3, non-small molecule mimetic compounds, inhibitors of GFAT, inhibitors of G6Pase, glucagon receptor antagonists, inhibitors of PEPCK, inhibitors of F-1,6-BPase, inhibitors of GP, RXR agonists, agonists of Beta-3 AR, PDHK inhibitors, inhibitors of gastric emptying and agonists of UCPs are disclosed in the patents and applications cited beyond each substance listed herein.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases. For example, the compounds to be combined can be present as a sodium salt, as a maleate or as a dihydrochloride. The active ingredient or a pharmaceutically acceptable salt thereof may also be used in form of a hydrate or include other solvents used for crystallization.

An antidiabetic compound, preferably selected from the group consisting of insulin signalling pathway modulators, like inhibitors of protein tyrosine phosphatases (PTPases), non-small molecule mimetic compounds and inhibitors of glutamine-fructose-6-phosphate amidotransferase (GFAT), compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK), pyruvate dehydrogenase kinase (PDHK) inhibitors, insulin sensitivity enhancers, insulin secretion enhancers, α-glucosidase inhibitors, inhibitors of gastric emptying, insulin, and α₂-adrenergic antagonists, or a pharmaceutically acceptable salt of such a compound, will be referred to hereinafter as COMBINATION PARTNER OF THE INVENTION.

A combined preparation which comprises a DPP-IV inhibitor in free or pharmaceutically acceptable salt form and at least one further COMBINATION PARTNER OF THE INVENTION and optionally at least one, i.e., one or more, e.g. two, pharmaceutically acceptable carrier for simultaneous, separate or sequential use is especially a “kit of parts” in the sense that the components, a DPP-IV inhibitor in free or pharmaceutically acceptable salt form and at least one further COMBINATION PARTNER OF THE INVENTION, can be dosed independently or by use of different fixed combinations with distinguished amounts of the components, i.e. at different time points or simultaneously. The parts of the kit of parts can then, e.g., be administered simultaneously or chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components. Preferably, there is at least one beneficial effect, e.g. a mutual enhancing of the effect of a DPP-IV inhibitor in free or pharmaceutically acceptable salt form, and at least one further COMBINATION PARTNER OF THE INVENTION, additional advantageous effects, less side effects, a combined therapeutical effect in a non-effective dosage of one or each of the components, and especially a synergism, e.g. a more than additive effect, between a DPP-IV inhibitor in free or pharmaceutically acceptable salt form, and at least one further COMBINATION PARTNER OF THE INVENTION.

The nature of conditions mediated by DPP-IV, especially diabetes, conditions of impaired fasting plasma glucose, and IGT, is multifactorial. Under certain circumstances, drugs with different mechanisms of action may be combined. However, just considering any combination of drugs having different mode of action but acting in the similar field does not necessarily lead to combinations with advantageous effects.

All the more surprising is the experimental finding that the combined administration of a DPP-IV inhibitor and at least one further COMBINATION PARTNER OF THE INVENTION results not only in a beneficial, especially a synergistic, therapeutic effect but also in additional benefits resulting from combined treatment such as a surprising prolongation of efficacy, a broader variety of therapeutic treatment and surprising beneficial effects on diseases and conditions associated, with diabetes, e.g. less gain of weight.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

It can be shown by established test models and especially those test models described herein that the combination of a DPP-IV inhibitor, especially (S)-1-{2-[5-cyanopyridin-2-yl)amino]ethyl-aminoacetyl}-2-cyano-pyrrolidine (DPP728) or (S)-1-[(3-hydroxy-1-adamantyl)amino]acetyl-2-cyano-pyrrolidine (LAF237), and at least one further COMBINATION PARTNER OF THE INVENTION results in a more effective prevention or preferably treatment of conditions mediated by DPP-IV, in particular diabetes, especially type 2 diabetes mellitus, conditions of impaired fasting plasma glucose, and conditions of IGT.

The person skilled in the pertinent art is fully enabled to select a relevant animal test model to prove the hereinbefore and hereinafter indicated therapeutic indications and beneficial effects. The pharmacological activity may, for example, be demonstrated following essentially an in-vivo test procedure in mice or in a clinical study as described hereinafter.

In-Vivo Test in Mice for Blood Glucose Control

ICR-CDI mice (male, five weeks old, body weight: about 20 g) are abstained from food for 18 hours, and then used as test subjects. The combination according to the present invention and the active ingredients alone are suspended in 0.5% CMC-0.14M sodium chloride buffer solution (pH 7.4). The solution thus obtained is administered orally in fixed volume amounts to the test subjects. After predetermined time, the percentage decrease of the blood glucose against the control group is determined.

Clinical double-blind, Randomized, Parallel-Group Study in Subjects with Type 2 Diabetes Mellitus Inadequately Controlled on Diet Alone

This study proves in particular the synergism of the claimed combined preparation or pharmaceutical composition, respectively. The beneficial effects on conditions mediated by DPP-IV, in particular type 2 diabetes mellitus can be determined directly through the results of this study or by changes in the study design which are known as such to a person skilled in the art.

The study is, in particular, suitable to compare the effects of monotherapy with a COMBINATION PARTNER OF THE INVENTION with those of a combination of DPP-IV inhibitor plus one of these compounds on glycemic control.

Subjects with a diagnosis of type 2 diabetes mellitus who have not achieved near normoglycemia (HbA_{1c}<6.8%) on diet only are chosen for this trial. The effects on glycemic control achieved with DPP-IV monotherapy, monotherapy with one COMBINATION PARTNER OF THE INVENTION, and the combination therapy of DPP-IV plus one COMBINATION PARTNER OF THE INVENTION are determined in this study after 24 weeks with the control achieved on placebo, all subjects continuing with the same diet as in the period before treatment. Measures of glycemic control are validated surrogate endpoints for the treatment of diabetes. HbA_{1c} is the single most reliable measurement for assessing glycemic control (D. Goldstein et al, Tests of Glycemia in Diabetes; Diabetes Care 1995, 18(6), 896-909) and is the primary response variable in this study. Since glycosylation of hemoglobin is determined by the glucose concentration at the time each red blood cell is made, HbA_{1c} provides an estimate of mean blood glucose for the previous three months.

Before starting with the double-blind treatment for 24 weeks, the subjects are administered for four weeks the placebos matching with the DPP-IV inhibitor, e.g. DPP728 and LAF237, before breakfast, lunch and dinner, and the placebos matching with one or more of the COMBINATION PARTNERS OF THE INVENTION (period I). For example, if the α-glucosidase inhibitors acarbose is chosen for the study, the placebo matching with acarbose is preferably administered together with the first bite of the meals taken for breakfast, lunch and dinner in period I. If the antidiabetic phenylacetic acid derivative repaglinide is chosen for the study, the placebos matching with repaglinide are preferably administered later on with breakfast, lunch and dinner in period I. If the antidiabetic thiazolidinedione troglitazone is chosen for the study, the placebos matching with troglitazone are preferably administered in period I with breakfast only. If the antidiabetic D-phenylalanine derivative nateglinide is chosen for the study, matching placebos are preferably administered before breakfast, lunch and dinner period I. If metformin is chosen for the study, matching placebos are preferably administered before breakfast and dinner.

The subjects are then separated into four treatment groups for the 24-week double-blind study (period II) as depicted in Tables 1 to 5 for the case that DPP728 is chosen as the DPP-IV inhibitor and one of the drugs comprising the antidiabetic thiazolidinedione troglitazone, the antidiabetic phenylacetic acid derivative repaglinide, the α-glucosidase inhibitor acarbose, the antidiabetic D-phenylalanine derivative nateglinide or the biguanide metformin is chosen as the combination partner.

Examples for Combinations to be Administered

TABLE 1
DPP728 plus troglitazone

DPP728 50 mg* + troglitazone placebo**
troglitazone 600 mg** + DPP728 placebo*
DPP728 50 mg* + troglitazone 600 mg**
DPP728 placebo* + troglitazone placebo**
*administered before breakfast, lunch, and dinner;
**administered once daily with breakfast

TABLE 2
DPP728 plus repaglinide

DPP728 50 mg* + repaglinide placebo*
repaglinide 1 mg* + DPP728 placebo*
DPP728 50 mg* + repaglinide 1 mg*
DPP728 placebo* + repaglinide placebo*
*administered before breakfast, lunch, and dinner

TABLE 3
DPP728 plus acarbose

DPP728 50 mg* + acarbose placebo**
acarbose 50 mg** + DPP728 placebo*
DPP728 50 mg* + acarbose 50 mg**
DPP728 placebo* + acarbose placebo**
*administered before breakfast, lunch, and dinner
**administered together with the first bite of breakfast, lunch and dinner

TABLE 4
DPP728 plus nateglinide

nateglinide (I) 120 mg* + DPP728 placebo*
DPP728 50 mg* + nateglinide (I) placebo*
nateglinide (I) 120 mg* + DPP728 50 mg*
nateglinide (I) placebo* + DPP728 placebo*
*administered before breakfast, lunch, and dinner

TABLE 5
DPP728 plus metformin

metformin 500 mg** + DPP728 placebo*
DPP728 50 mg* + metformin placebo**
metformin 500 mg** + DPP728 50 mg*
metformin placebo** + DPP728 placebo*
*administered before breakfast, lunch, and dinner
**administered before breakfast and dinner

DPP728 tablets contain either 50 mg of the compound or matching placebo. Nateglinide tablets contain either 120 mg or matching placebo. Troglitazone 200 mg tablets, repaglinide 1 mg tablets, acarbose 50 mg tablets and metformin 500 mg tablets can be purchased commercially and overencapsulated to match the corresponding placebo capsules.

The subjects are then separated into four treatment groups for the 24-week double-blind study (period II) as depicted in Table 1. Approximately 170 subjects are randomized per treatment group. The total study duration including the run-in period for each subject is 28 weeks. Statistical analysis can be carried out by methods known in the art.

The subject is advised not to take the morning dose of study medication or eat breakfast on the day of a scheduled study visit. The morning dose is administered by site personnel after the collection of all fasting laboratory samples and completion of all study procedures. Visits are scheduled to be performed at 2 week intervals during period I, and 4 to 8 week intervals during period II. Subjects have fasted for at least 7 hours at the time of each visit. All blood samples for laboratory evaluations are drawn between 7:00 AM and 10:00 AM. All tests are conducted in accordance with Good Laboratory Practice principles following procedures known in the art.

HbA_{1c} is measured by High Performance Liquid Chromatography (HPLC) using the ion-exchange method on a Bio-Rad Diamat analyzer. A back-up affinity method are used if hemoglobin variants or hemoglobin degradation peaks are observed.

Further parameters to be determined are fasting plasma glucose (FPG), fasting lipids (total, HDL (high density lipoprotein)- and LDL (low density lipoprotein)-cholesterol, and triglycerides) and body weight. FPG will be measured using the hexokinase method and LDL-cholesterol will be calculated using the Friedewald formula if triglycerides are <400 mg/dL (4.5 mmol/l).

Various parameters of the study described above can be modified, e.g. in order to optimize the dosage for special diseases or indications mentioned herein, to cope with tolerability problems during the study or to obtain similar or identical results with less efforts. For example, a different subject population can be involved in such a clinical trial, e.g. subjects with a diagnosis of type 2 diabetes mellitus who have achieved near normoglycemia (HbA_{1c}<6.8%) on diet alone, subjects with diseases other than diabetes mellitus, e.g. other metabolic disorders, or subjects selected by other criteria, such as age or sex; the subject number can be decreased, e.g. to a number of between 70 and 150, especially 100 or 120, subjects per treatment group; treatment groups (listed exemplary in Table 1) can be deleted, i.e. for example to carry out a study with a comparison of the combination of a DPP-IV inhibitor and at least one further COMBINATION PARTNER OF THE INVENTION versus a DPP-IV inhibitor alone; the term of the placebo run-in period (period I) can be changed, i.e. it can be extended, shortened or deleted; the visit schedule can be extended, e.g. to every 10, 12 or 14 weeks; the visit instructions can be changed, e.g. the instruction that blood samples for laboratory evaluations have to be drawn between 7:00 AM and 10:00 AM; HbA_{1c} can be determined by other means; or one or more of the parameters to be determined during the study mentioned above, e.g. FPG or fasting lipids, can be deleted or the determination of additional parameters (see below) can be added.

Additional parameters can be determined in the course of the study, e.g. by additional tests. Such additional tests can comprise the analysis of body liquids in order to determine amounts or numbers for parameters such as those listed below and can serve e.g. the purpose of determining the tolerability of the administered active ingredients: determination of hematocrit and hemogloblin, platelet count, erythrocyte count, total and differential leukocyte count (basophils, eosinophils, lymphocytes, monocytes, segmented neutrophils and total neutrophils); determination of albumin, alkaline phosphatase, alanine amino transferase (serum glutamic pyruvic transaminase), aspartate amino transferase (serum glutamic oxaloacetic transaminase), blood urea nitrogen or urea, bicarbonate, calcium, chloride, total creatine phosphokinase (CPK), creatine phosphokinase muscle-brain fraction isoenzyme (if CPK is elevated), direct bilirubin, creatinine, γ-glutamyl transferase, lactate dehydrogenase, potassium, sodium, total bilirubin, total protein and uric acid in the blood; determination of bilirubin, glucose, ketones, pH, protein, and specific gravity in the subjects urine; determination of body weight, blood pressure (systolic and diastolic, after 3 minutes sitting) and radial pulse (after 3 minutes sitting).

The results of the studies show that the combination according to the present invention can be used for the prevention and preferably the treatment of conditions mediated by DPP-IV, in particular type 2 diabetes mellitus. The combination of the present invention can also be used for the prevention and preferably the treatment of other condition mediated by DPP-IV.

Furthermore, in a number of combinations as disclosed herein the side-effects observed with one of the components surprisingly do not accumulate on application of the combination.

Preferably, the jointly therapeutically effective amounts of a DPP-IV inhibitor in free or pharmaceutically acceptable salt form and at least one further pharmaceutically active compound are administered simultaneously or sequentially in any order, separately or in a fixed combination.

The condition mediated by DPP-IV is preferably selected from the group consisting of diabetes, impaired fasting plasma glucose, impaired glucose tolerance, metabolic acidosis, ketosis, arthritis, obesity and osteoporosis.

Very preferably, the condition mediated by DPP-IV is type 2 diabetes mellitus.

It is one objective of this invention to provide a pharmaceutical composition comprising a quantity, which is jointly therapeutically effective against conditions mediated by DPP-IV, in particular diabetes, more especially-type 2 diabetes mellitus, conditions of impaired fasting plasma glucose, and conditions of IGT, of a DPP-IV inhibitor (i) or a pharmaceutically acceptable salt thereof and (ii) at least one further COMBINATION PARTNER OF THE INVENTION and at least one pharmaceutically acceptable carrier.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

The novel pharmaceutical preparations contain, for example, from about 10% to about 100%, e.g., 80% or 90%, preferably from about 20% to about 60%, of the active ingredient. Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

In this composition, components (i) and (ii) can be administered together, one after the other or separately in one combined unit dose form or in two separate unit dose forms. In one preferred embodiment of the invention, the unit dose form is a fixed combination. In a fixed combination the components (i) and (ii) are administered in the form of a single galenic formulation, e.g. a single tablet or a single infusion.

A further aspect of the present invention is the use of a pharmaceutical composition comprising a DPP-IV inhibitor and at least one further COMBINATION PARTNER OF THE INVENTION, in each case in free form or in form of a pharmaceutically acceptable salt thereof for the preparation of a pharmaceutical preparation for the prevention or treatment of conditions mediated by DPP-IV, in particular diabetes, more especially type 2 diabetes mellitus, conditions of impaired fasting plasma glucose, and conditions of IGT.

A therapeutically effective amount of each of the components of the combination of the present invention may be administered simultaneously or sequentially and in any order, and the components may be administered separately or as a fixed combination. For example, the method of treatment of the invention may comprise (i) administration of a DPP-IV inhibitor in free or pharmaceutically acceptable salt form and (ii) administration of at least one further COMBINATION PARTNER OF THE INVENTION simultaneously or sequentially in any order, in jointly therapeutically effective amounts, preferably in synergistically effective amounts, e.g. in daily dosages corresponding to the ratios described herein.

The corresponding active ingredient or a pharmaceutically acceptable salt thereof may also be used in form of a hydrate or include other solvents used for crystallization.

Furthermore, the term administering also encompasses the use of prodrugs of any of the anti-diabetic drugs that convert in vivo to the selective anti-diabetic drug. The instant invention is therefore to be understood as embracing all such regimes of simultaneous or alternating treatment and the term “administering” is to be interpreted accordingly.

If the combination comprises nateglinide, a composition, in particular a pharmaceutical composition, comprising solely nateglinide can be produced by a process that comprises granulating in the presence of water to form granules, drying the granules, and optionally screening the granules, for example, through a wire mesh screen. All of the ingredients of the composition may be added prior to or during the granulation. Alternatively, all or a portion of one or more of the ingredients may be added after the granulation step is complete. For example, all or a portion of anti-adherent (e.g., silica), all or a portion of lubricant (e.g., magnesium stearate) and/or all or a portion of disintegrant (e.g., croscarmellose or any salt thereof) may be added after the granulation. In one aspect of the invention, all ingredients except the magnesium stearate and the colloidal silica are loaded into the granulator, then they are added later. The process of producing this composition, in particular pharmaceutical composition, may be performed without the need for a pulverization step. As used herein, the terms “pulverization” and “pulverize” refer to any process that involves the grinding or smashing cutting of particles to reduce the particles' size. The composition, in particular pharmaceutical composition, is capable of being produced without pulverizing the granules between the granulation step and the drying and/or compression step used to form the granules into a tablet. In one preferred embodiment of the invention, nateglinide is used in the B-type or H-type crystal modification.

The invention relates in particular to a commercial package comprising jointly therapeutically effective amounts of a DPP-IV inhibitor, in free or pharmaceutically acceptable salt form, and at least one further COMBINATION PARTNER OF THE INVENTION together with instructions for use thereof in the treatment of conditions mediated by DPP-IV, in particular diabetes, more especially type 2 diabetes mellitus, conditions of impaired fasting plasma glucose, and conditions of IGT.

A further aspect of the present invention is a method of treating a condition mediated by DPP-IV, in particular type 2 diabetes mellitus, comprising administering to a warm-blooded animal in need thereof jointly therapeutically effective amounts of a DPP-IV inhibitor in free or pharmaceutically acceptable salt form, and at least one further COMBINATION PARTNER OF THE INVENTION. Preferably, in this method of treating the active ingredients are administered simultaneously or sequentially in any order, separately or in a fixed combination. In one preferred embodiment of such method the jointly therapeutically effective amounts of a dipeptidylpeptidase-IV inhibitor in free or pharmaceutically acceptable salt form and at least one further COMBINATION PARTNER OF THE INVENTION are provided as a combined preparation.

Furthermore, the present invention provides a method of treating conditions of impaired glucose tolerance and impaired fasting plasma glucose comprising administering to a warm-blooded animal in need thereof jointly therapeutically effective amounts of a DPP-IV inhibitor in free or pharmaceutically acceptable salt form, and at least one further COMBINATION PARTNER OF THE INVENTION.

Furthermore, the invention relates to a method of improving the bodily appearance of a mammal which comprises orally administering to said mammal, including man, especially man suffering from a metabolic disorder, in particular type 2 diabetes, a combined preparation or pharmaceutical composition described herein in a dosage effective to influence, e.g., to increase or decrease, the glucose metabolism, or to influence the body weight by other mechanisms, and repeating said dosage until a cosmetically beneficial loss of body weight has occurred. Such combinations described herein can also be used to prevent, for cosmetic reasons, a further increase in body weight in humans experiencing such an increase. Moreover, the invention relates to the combinations described herein useful for improving the bodily appearance of a mammal, especially a human being, and the use of such combinations in order to improve the bodily appearance of a mammal, especially a human being. Overweight is one of the risk factors for developing a metabolic disorder, in particular type 2 diabetes, and at the same time often the result of such a metabolic disorder, especially type 2 diabetes. Furthermore, a number of antidiabetics are known to cause weight gain. Hence, humans suffering from metabolic disorders, especially type 2 diabetes, are often faced with overweight. Therefore, the cosmetically beneficial loss of body weight can be effected especially in humans suffering from a metabolic disorder, such as type 2 diabetes. The combinations described herein can also be used to replace or complement an antidiabetic drug taken by a human suffering from type 2 diabetes in order to prevent, for cosmetic reasons, a further increase of the body weight.

The dosage range of the combination of a DPP-IV inhibitor and at least one further COMBINATION PARTNER OF THE INVENTION to be employed depends upon factors known to the person skilled in the art including species of the warm-blooded animal, body weight and age, the nature and severity of the condition to be treated, the mode of administration and the particular substance to be employed. Unless stated otherwise herein, the DPP-IV inhibitor and at least one further COMBINATION PARTNER OF THE INVENTION are preferably divided and administered from one to four times per day.

The weight ratio of the daily doses of DPP728 or LAF237 or a pharmaceutically acceptable salt thereof to at least one further COMBINATION PARTNER OF THE INVENTION may vary within wide limits depending in particular on the needs of the warm-blooded animal treated.

In one preferred embodiment of the invention the following weight ratios of DPP728 or LAF237 or a pharmaceutically acceptable salt thereof to one of the indicated further COMBINATION PARTNERS OF THE INVENTION should be administered in order to obtain a synergistic effect:

TABLE 6

| | |
|---|---|
| Inhibitors of PTPases | between 200:1 and 1:50, preferably between |
| | 100:1 and 1:25 |
| Inhibitors of GSK-3 | between 200:1 and 1:50, preferably between |
| | 100:1 and 1:25 |
| Inhibitors of G6Pase | between 200:1 and 1:50, preferably between |
| | 100:1 and 1:25 |
| Inhibitors of PEPCK | between 200:1 and 1:50, preferably between |
| | 100:1 and 1:25 |
| Inhibitors of F1, 6Bpase | between 200:1 and 1:50, preferably between |
| | 100:1 and 1:25 |
| Inhibitors of GP | between 200:1 and 1:50, preferably between |
| | 100:1 and 1:25 |
| RXR agonists | between 200:1 and 1:50, preferably between |
| | 100:1 and 1:25 |
| Agonists of Beta-3 AR | between 200:1 and 1:50, preferably between |
| | 100:1 and 1:25 |
| UCP agonists | between 200:1 and 1:50, preferably between |
| | 100:1 and 1:25 |

In a more preferred embodiment of the invention the following weight ratios of DPP728 or LAF237 or a pharmaceutically acceptable salt thereof to one of the indicated further COMBINATION PARTNERS OF THE INVENTION should be administered in order to obtain a synergistic effect of the components:

TABLE 7

| further | |
|---|---|
| pharmaceutically active | DPP728 or LAF237/further pharmaceutically |
| compound | active compound |
| Nateglinide | Between 200:1 and 1:48, |
| | Preferably between 12:1 and 1:5, |
| | e.g. 1:1 |
| Acarbose | between 20:1 and 1:24, |
| | preferably between 2:1 and 1:2, |
| | e.g. 1:1 |
| Troglitazone | between 1:1 and 1:10, |
| | preferably between 1:2 and 1:6, |
| | e.g. 1:4 |
| Metformin | between 4:1 and 1:60, |
| | preferably between 1:1 and 1:10, |
| | e.g. 1:6 |
| Repaglinide | between 100:1 and 15:1, |
| | preferably between 60:1 and 20:1, |
| | e.g. 50:1 |
| Inhibitors of PTPases | between 200:1 and 1:50, preferably between |
| | 100:1 and 1:25 |
| Inhibitors of GSK-3 | between 200:1 and 1:50, preferably between |
| | 100:1 and 1:25 |
| Inhibitors of G6Pase | between 200:1 and 1:50, preferably between |
| | 100:1 and 1:25 |
| Inhibitors of PEPCK | between 200:1 and 1:50, preferably between |
| | 100:1 and 1:25 |
| Inhibitors of F1, 6Bpase | between 200:1 and 1:50, preferably between |
| | 100:1 and 1:25 |
| Inhibitors of GP | between 200:1 and 1:50, preferably between |
| | 100:1 and 1:25 |
| RXR agonists | between 200:1 and 1:50, preferably between |
| | 100:1 and 1:25 |
| Agonists of Beta-3 AR | between 200:1 and 1:50, preferably between |
| | 100:1 and 1:25 |
| UCP agonists | between 200:1 and 1:50, preferably between |
| | 100:1 and 1:25 |

If the warm-blooded animal is a human of about 70 kg body weight the dosages of the at least one further pharmaceutically active compounds are preferably the following:

TABLE 8

| pharmaceutically active | | |
|---|---|---|
| compound | preferred dosage | most preferred dosage |
| acarbose | about 50 to 600 mg/day | about 150 to 300 mg/day |
| AD-5075 | about 0.1 to 2500 mg/day | about 1 to 1000 mg/day |
| AY-31637 | about 0.5 to 200 mg/kg body | 2.5 to 100 mg/kg body |
| | weight of the patient per day | weight of the patient per day |
| ciglitazone | about 0.25 to 200 mg/kg | about 0.5 to 50 mg/kg body |
| | body weight of the patient | weight of the patient per day |
| | per day | |
| darglitazone | about 0.05 to 50 mg/kg body | about 0.05 to 5 mg/kg body |
| | weight of the patient per day | weight of the patient per day |
| DN-108 | about 0.25 to 200 mg/kg | about 5 to 100 mg/kg body |
| | body weight of the patient | weight of the patient per day |
| | per day | |
| DPP728 | about 25 to 1000 mg/day | about 150 to 300 mg/day |
| englitazone | about 0.05 to 50 mg/kg body | about 0.05 to 5 mg/kg body |
| | weight | weight |
| glibenclamide | about 0.1 to 25 mg/day | about 1.75 to 10.5 mg/day |
| glibornuride | about 5 to 150 mg/day | about 12.5 to 75 mg/day |
| gliclazide | about 20 to 480 mg/day | about 80 to 240 mg/day |
| glimepiride | about 0.25 to 12 mg/day | about 1 to 6 mg/day |
| gliquidone | about 5 to 250 mg/day | about 30 to 120 mg/day |
| glisoxepid | about 0.5 to 25 mg/day | about 2 to 16 mg/day |
| incretin hormone like GLP-1 | about 20 to about 100 μg per | |
| | day | |
| KRP297 | about 0.1 to 2500 mg/day | about 1 to 1000 mg/day |
| MCC555 | about 0.1 to 2000 mg/day | about 0.5 to 100 mg/day |
| metformin | about 250 to 1500 mg/day | about 500 to 1250, e.g. |
| | | 1000, mg/day |
| miglitol | about 50 to 500 mg/day | about 100 to 300 mg/day |
| nateglinide | about 5 to 1200 mg/day | about 25 to 800 mg/day |
| pioglitazone | about 0.1 to 1000 mg/day | about 10 to 150, for example |
| | | 15, 30, 45 or 90, mg/day |
| repaglinide | about 0.5 to 16 mg/day | about 1 to 8 mg/day |
| rosiglitazone | about 0.1 to 500 mg/day | about 1 to 20, for example 1, |
| | | 2, 4 or 8, mg/day |
| T-174 | about 0.1 to 2500 mg/day | about 1 to 1000 mg/day |
| tolbutamide | about 250 to 3000 mg/day | about 1000 to 2000 mg/day |
| troglitazone | about 0.1 to 2000 mg/day | about 50 to 1000 for |
| | | example 100, 200, 400, 600 |
| | | or 800, mg/day, mg/day |
| 5-[3-(4-chlorophenyl])-2- | about 0.1 to 2500 mg/day | about 1 to 1000 mg/day |
| propynyl]-5-phenylsulfonyl)- | | |
| thiazolidine-2,4-dione | | |
| 5-[3-(4-chlorophenyl])-2- | about 0.1 to 2500 mg/day | about 1 to 1000 mg/day |
| propynyl]-5-(4-fluoro- | | |
| phenylsulfonyl)thiazolidine- | | |
| 2,4-dione | | |
| N-(N′-substituted glycyl)-2- | about 0.1 to 250 mg/kg body | about 1 to 100 mg/kg body |
| cyanopyrrolidine of formula I | weight of the patient per day | weight of the patient per day |

The following Examples shall illustrate the invention described above; they are not, however, intended to limit the scope of the invention in any way.

### EXAMPLE 1

### Tablets of Nateglinide

108,000 tablets, each which contain 120 mg of nateglinide are prepared as follows:

| | | | |
|---|---|---|---|
| Composition: | nateglinide | 12.960 | kg |
| | lactose, NF | 30.564 | kg |
| | microcrystalline cellulose, NF | 15.336 | kg |
| | povidone, USP | 2.592 | kg |
| | croscarmellose sodium, NF | 3.974 | kg |
| | colloidal silicon dioxide, NF | 1.382 | kg |
| | magnesium stearate, NF | 1.231 | kg |
| | coating: opadry yellow | 1.944 | kg |

| | |
|---|---|
| purified water, USP* | Q.S. |

*removed during process

Preparation process: The microcrystalline cellulose, povidone, part of the croscarmellose sodium, nateglinide and lactose are mixed in a high shear mixer and afterwards granulated using purified water. The wet granules are dried in a fluid bed dryer and passed through a screen. The colloidal silicon dioxide and the rest of the croscarmellose sodium are mixed, passed through a screen and blended with the dried granules in a V-blender. The magnesium stearate is passed through a screen, blended with the blend from the V-blender and afterwards the total mixture is compressed to tablets. The opadry yellow is suspended in purified water and the tablets are coated with the coating suspension.

### EXAMPLE 2

### Galenic Formulation of Nateglinide No. 1

intra-granular:

| | | |
|---|---|---|
| nateglinide | 120 | mg |
| lactose monohydrate | 283 | mg |
| microcrystalline cellulose | 142 | mg |
| povidone | 24 | mg |
| croscarmellose sodium | 24 | mg |

extra-granular:

| | | |
|---|---|---|
| magnesium stearate | 7 | mg |
| opadry white | 20 | mg |

### EXAMPLE 3

### Galenic Formulation of Nateglinide No. 2

intra-granular:

| | | |
|---|---|---|
| nateglinide | 120 | mg |
| lactose monohydrate | 283 | mg |
| microcrystalline cellulose | 142 | mg |
| povidone | 24 | mg |
| croscarmellose sodium | 24 | mg |

extra-granular:

| | | |
|---|---|---|
| croscarmellose sodium | 12.8 | mg |
| magnesium stearate | 11.4 | mg |
| opadry yellow | 18.0 | mg |
| colloidal silicon dioxide | 12.8 | mg |

### EXAMPLE 4

### Tablets of Nateglinide

108,000 tablets, each which contain 120 mg of nateglinide are prepared as follows:

| | | | |
|---|---|---|---|
| Composition: | nateglinide | 12.960 | kg |
| | lactose, NF | 30.564 | kg |
| | microcrystalline cellulose, NF | 15.336 | kg |
| | povidone, USP | 2.592 | kg |
| | croscarmellose sodium, NF | 3.974 | kg |
| | colloidal silicon dioxide, NF | 1.382 | kg |
| | magnesium stearate, NF | 1.231 | kg |
| | coating: opadry yellow | 1.944 | kg |

| | |
|---|---|
| purified water, USP* | Q.S. |

*removed during process

Preparation process: The microcrystalline cellulose, povidone, a portion of the croscarmellose sodium, nateglinide and lactose are granulated in a collette gral granulator with the addition of purified water. The wet granules are dried in a fluid bed dryer and passed through a screen. The colloidal silicon dioxide and the rest of the croscarmellose sodium are mixed, passed through a screen and blended with the dried granules in a V-blender. The magnesium stearate is passed through a screen, blended with the blend from the V-blender and afterwards the total mixture is compressed to tablets. The opadry yellow is suspended in purified water and the tablets are coated with the coating suspension. Variants of this process include adding the colloidal silica and the remaining croscarmellose sodium to the second granulator load after drying, then screening together; and combining as many as 3 granulator/drier loads per batch.

### EXAMPLE 5

### Pharmaceutical Composition of Nateglinide (120 mg)

| | | |
|---|---|---|
| nateglinide | 120 | mg |
| lactose monohydrate | 283 | mg |
| microcrystalline cellulose | 142 | mg |
| Povidone | 24 | mg |
| croscarmellose sodium | 36.8 | mg |
| magnesium stearate | 11.4 | mg |
| opadry yellow | 18.0 | mg |
| colloidal silicon dioxide | 12.8 | mg |

All references, including U.S., World and EP Patents and applications referred to herein are hereby incorporated by reference in their entirety as if set forth in full herein.

1. A pharmaceutical composition comprising a dipeptidylpeptidase-IV (DPP-IV) inhibitor (S)-1-[(3-hydroxy-1-adamantyl)amino]acetyl-2-cyano-pyrrolidine, in free or pharmaceutically acceptable salt form, and at least one further anti-diabetic compound selected from the group consisting of anti-diabetic thiazolidinediones (glitazones), non-glitazone type peroxisome proliferator-activated receptor γ (PPARγ) agonists and dual PPARγ/PPARα agonists, or in each case, a pharmaceutically acceptable salt thereof, and optionally at least one pharmaceutically acceptable carrier.

2. The pharmaceutical composition of claim 1 wherein the anti-diabetic compound is an anti-diabetic thiazolidinedione.

3. The pharmaceutical composition of claim 2 wherein the anti-diabetic thiazolidinedione is selected from the group consisting of pioglitazone, rosiglitazone and troglitazone.

4. The pharmaceutical composition of claim 1 wherein the anti-diabetic compound is a non-glitazone type peroxisome proliferator-activated receptor γ (PPARγ) agonist.

5. The pharmaceutical composition of claim 4 wherein the non-glitazone PPARγ agonist is a N-(2-benzoylphenyl)-L-tyrosine analogue selected from GI-262570 (farglitazar) and JTT501 (reglitazar).

6. A method of treating diabetes, obesity, or conditions of impaired glucose tolerance (IGT) in a patient comprising administering a therapeutically effective amount of the pharmaceutical composition according to claim 1.
